(19) United States
(12) Patent Application Publication
(10) Pub. No.: US 2026/0283469 A1
(43) Pub. Date: Sep. 24, 2026

(54) METHOD OF MONITORING INTAKE OF BREAST MILK BY AN INFANT

(71) Applicant: Britta Christiansen, Gerlingen (DE)

(72) Inventor: Volkan Görkem Büyükyildiz, Gerlingen (DE)

(73) Assignee: Britta Christiansen, Gerlingen (DE)

(21) Appl. No.: 18/874,306

(22) PCT Filed: Jun. 22, 2022
(86) PCT No.: PCT/EP2022/067028

§ 371 Date: Dec. 12, 2024

(51) Int. Cl. A61B 5/00

(52) U.S. Cl. CPC A61B 5/0075; A61B 5/0082; A61B 5/6822; A61B 5/7246; A61B 5/7271; A61B 2503/04; A61B 2560/0462

(57) ABSTRACT

Problem: While a comparison of pre-nursing to post-nursing weight of the breastfed child tends to yield imprecise results, the extraction of breast milk into a container may impair some of the benefits commonly associated with breastfeeding, for example, improved maternal bonding as well as oral and cognitive development of the infant. Solution: Method of monitoring intake of breast milk by an infant in a collar (10), comprising: transmitting light from light sources (L1-L4) through the infant's esophagus, receiving the transmitted light at photosensors (P) preferably arranged diametrically opposite the light sources (L1-L4) with respect to the esophagus, and, based on the light received, determining the intake, for example, in terms of volume, mass, or energy.

### TECHNICAL FIELD

The invention relates to the products and process as per the first portion of the independent claims.

### BACKGROUND ART

The American Academy of Pediatrics (AAP) recommends exclusive breastfeeding for about the first six months of life, with continued breastfeeding through twelve months and beyond as appropriate complementary foods are introduced. The World Health Organization (WHO) similarly recommends that infants be exclusively breastfed for the first six months, with breastfeeding continuing for two or more years as safe and nutritionally adequate complementary foods are added.

NPL 1: identifies perceived insufficient milk (PIM) as a major barrier to successful breastfeeding, that is, the belief of the nursing mother that her milk be insufficient for her infant's needs. State-of-the-art methods for mitigating PIM encompass reinforcing appropriate early infant feeding routines and closely monitoring breastfeeding dyads during the first postpartum weeks to ensure that breastfeeding is well established.

In terms of technical measures, frequent infant weight checks and periodic test weighing (comparing pre-nursing to post-nursing weight) have been proposed to provide concrete evidence for breastfeeding mothers concerned about the adequacy of milk intake by their nursling. Alternatively, breast pumps—such as disclosed in PTL1—may be employed to extract and direct the milk into a container where its volume can be easily measured prior to bottle-feeding.

PTL 2: discloses the subject matter of the first portion of claim 1.

An apparatus for determining the amount of human milk supplied to a feeding baby during a breast-feeding session is disclosed in PTL3. The apparatus comprises a feeding cap adapted to be mounted on the nipple region of a breast of a mother. The cap defines an outlet through which milk passes to the feeding baby. A flowmeter is provided to measure the amount of milk passing through the outlet.

PTL 4: discloses reflection spectroscopy to determine milk intake in the stomach.

### SUMMARY OF INVENTION

The present invention enables percutaneous determination of breast milk intake during breastfeeding using an optical method.

### Technical Problem

While a comparison of pre-nursing to post-nursing weight of the breastfed child tends to yield imprecise results, the extraction of breast milk into a container may impair some of the benefits commonly associated with breastfeeding, for example, improved maternal bonding as well as oral and cognitive development of the infant. Both methods are considered laborious and time-consuming.

### Solution to Problem

The problem is solved as per the second portion of claim 1.

### Advantageous Effect of Invention

The present invention addresses the need for a rapid and reliable measurement method whereby the amount of milk consumed during a breastfeeding session can be determined conveniently and non-invasively. Consequently, any nutritional deficiencies on the part of the nursling may be detected and compensated without delay.

### BRIEF DESCRIPTION OF DRAWINGS

FIG. 1 is a perspective view of an infant being breastfed.

FIG. 2 is a longitudinal section of a collar worn around the infant's neck.

FIG. 3 is a cross section of the collar.

### DESCRIPTION OF EMBODIMENTS

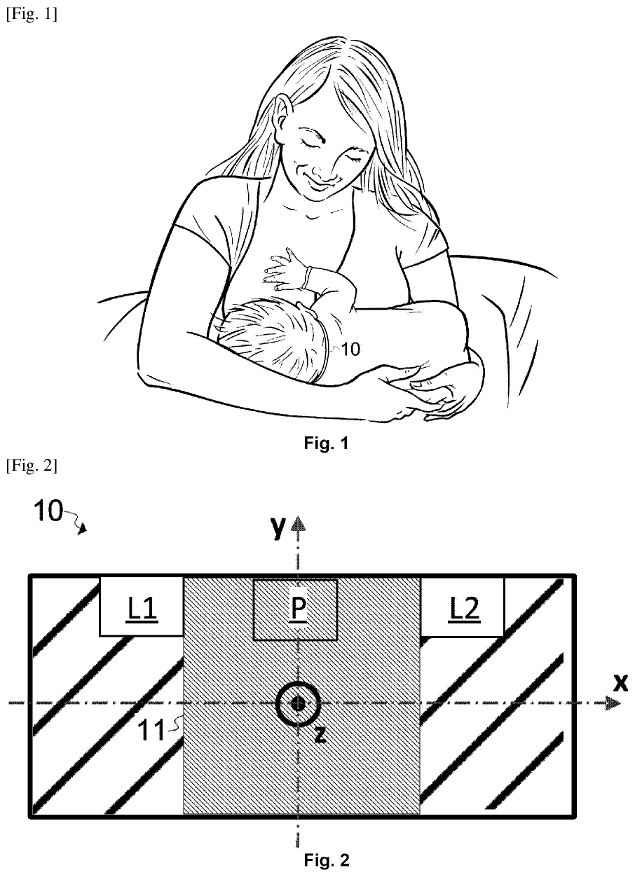

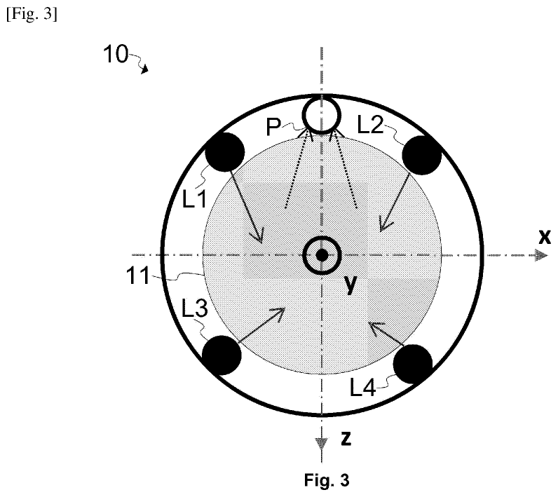

FIG. 1 illustrates domestic use of the collar (10) depicted in [FIG. 2] and [FIG. 3]. Comparable to a pulse oximeter, it measures the flow rate or amount of milk imbibed percutaneously at the infant's neck (11) based on differences in optical interference. To this end, upon transillumination of the neck (11) at specific wavelengths, the mass or molar attenuation coefficient of breast milk, which is perceived as white to yellow, may be used to distinguish such fluid from other bodily chemical species.

For this purpose, four LEDs (L1, L2, L3, L4) or laser diodes serve as optical excitation sources that are activated sequentially. Absorption of the emitted light within the neck (11) is translated into electric current using a photodiode (P) as a receiver. To minimize external interference, the periphery of the transmitters (L1, L2, L3, L4) and receiver (P) is screened off by the shielding collar (10).

For correcting any superficial variance, such as in terms of thickness of the skin tissue, a relative rather than an absolute measure is applied. Hence, in a preliminary step, the maximum and minimum flow rate achievable by an infant is related to light intensity as a function of the attainable wavelengths. Hereinafter, this measure shall be referred to as the fractional milk flow rate. Such fractional flow rate will be proportional to the total flow rate.

An established ratio between fractional and total flow rate may be reflected constructively in the photodiode's gain such that the electrical current output by the photodiode (P) relates directly to the total flow rate, although such relation should not be considered essential to the method at hand. Alternatively, total flow rate can be retrieved from a lookup table.

Moreover, a transceiver having an antenna for transmission and reception may be provided for communications, such as via a cellular network, WLAN, WPAN, or by cable. Suitable transmission protocols include TCP/IP. It is well understood that any suitable transmission medium and protocol may be used for this purpose. Communications may be compressed as required.

For optimal approximation of effective milk intake, various numbers and combinations of diodes (L1-L4, P) with regard to wavelength, arrangement, and scattering angle are conceivable without departing from the scope of the present invention. Likewise, a computerized assistant, including but not limited to a smartphone application, may be provided to evaluate and optimize the nursing routine.

### INDUSTRIAL APPLICABILITY

The invention is applicable, inter alia, throughout the medical equipment industry.

### REFERENCE SIGNS LIST

| | |
|---|---|
| 10 | Collar |
| 11 | Infant's neck |
| L1 | White LED |
| L2 | Yellow LED |
| L3 | Red LED |
| L4 | Infrared LED |
| P | Photodiode |

### CITATION LIST

The following documents are cited hereinbefore.

### PATENT LITERATURE

PTL 1: U.S. Pat. No. 1,644,257 A (EDWARD LASKER) 10 Apr. 1927
PTL 2: CN 110338757 A (HANGZHOU CHILDRENS HOSPITAL) 18 Oct. 2019
PTL 3: WO 01/54488 A (VASLOV TRADERS PROPRIETARY LTD) 2 Aug. 2001
PTL 4: US2019125244 A (MOLKENTHIN BRITTANY) 2 May 2019

### Non-Patent Literature

NPL 1: NEIFERT, Marianne, et al. Overcoming clinical barriers to exclusive breastfeeding. Pediatric Clinics. 2013, vol. 60, no. 1, p. 115-145.

1. A method of monitoring intake of breast milk by an infant in a collar, comprising:
calculating an intake in terms of volume, mass, or energy, from a flow rate of milk through a cross-sectional area of an infant's esophagus, characterized in transmitting light from light sources through the esophagus,
receiving the transmitted light at photosensors, and,
based on the light received, determining the intake by estimating the flow rate by means of absorption spectroscopy.

2. The method as per claim 1, wherein the flow rate is estimated by comparing intensity of the light received at the photosensors with predetermined reference intensities associated with a minimum and maximum flow rate.

3. The method as per claim 2, wherein the estimated flow rate is retrieved from a lookup table based on the intensity of the light received.

4. The method as per claim 1, wherein photocurrent through the photosensors is amplified into an output current linearly dependent on the flow rate.

5. The method as per claim 1, wherein the light sources are on the collar and the light is visible or infrared.

6. The method as per claim 5, wherein three among the light sources emit monochromatically at wavelengths of 575 nm, 700 nm, and 910 nm, respectively.

7. The method as per claim 5, wherein one among the light sources emits white light.

8. The method as per claim 1, wherein the light sources are on the collar and the light sources are operated alternately.

9. A collar, comprising:
light sources,
photosensors, and
means adapted to execute the method of claim 1.

10. The collar as per claim 9, wherein the light sources and the photosensors are semiconductor diodes.

11. The collar as per claim 9, wherein the light sources are laser diodes.

12. A computer program, comprising:
instructions to execute the method of claim 1 when run on a processor connected to a collar comprising light sources and photosensors and means adapted to execute the method.

13. A data carrier signal carrying the computer program of claim 12.

14. The method as per claim 1, wherein the light sources are on the collar.

15. The method as per claim 1, wherein the photosensors are on the collar.

16. The method as per claim 1, wherein the photosensors are arranged diametrically opposite the light sources with respect to the esophagus.

17. The collar as per claim 9, wherein the collar is opaque to ambient light.

18. The collar as per claim 9, wherein at least one of the light sources is diametrically opposed to at least one of the photosensors.

19. The method as per claim 2, wherein a photocurrent through the photosensors is amplified into an output current linearly dependent on the flow rate.

20. The method as per claim 14, wherein the light sources are operated sequentially.
